(19) United States
(12) Patent Application Publication
(10) Pub. No.: US 2025/0255494 A1
(43) Pub. Date: Aug. 14, 2025

(54) Hearing Devices, Systems, and Methods for Measuring a Core Body Temperature of a User

(71) Applicant: SONOVA AG, Staefa (CH)

(72) Inventors: Maryam Mostafaei, Wallisellen (CH); Samuel Elia Johannes Knobel, Zurich (CH); Hans-Ueli Roeck, Hombrechtikon (CH); Konstantin Silberzahn, Zürich (CH)

(21) Appl. No.: 18/439,901

(22) Filed: Feb. 13, 2024

(51) Int. Cl. A61B 5/0205; A61B 5/00; A61B 5/024; A61B 5/11; G01J 5/00; G01J 5/80; G01P 13/00

(52) U.S. Cl. CPC A61B 5/02055; A61B 5/1118; A61B 5/6817; G01J 5/0025; G01J 5/80; G01P 13/00; A61B 5/02438; A61B 2560/0252; A61B 2562/0271; A61B 2562/04

(57) ABSTRACT

An exemplary hearing device may be configured to be inserted at least partially within an ear canal of a user of the hearing device. The hearing device may include a first temperature sensor configured to detect ear canal temperature within the ear canal, a second temperature sensor configured to detect ambient temperature outside of the ear canal, and a processor. The processor may be configured to determine a work status of the user indicative of an activity level of the user and determine a core body temperature of the user based on the ear canal temperature detected by the first temperature sensor, the ambient temperature detected by the second temperature sensor, and the work status of the user.

### BACKGROUND INFORMATION

Hearing devices (e.g., hearing aids) are used to improve the hearing capability and/or communication capability of users of the hearing devices. Such hearing devices are configured to process a received input sound signal (e.g., ambient sound) and provide the processed input sound signal to the user (e.g., by way of a receiver (e.g., a speaker) placed in the user's ear canal or at any other suitable location).

In addition to being used to facilitate providing the processed input sound signal to the user, hearing devices may include one or more sensors configured to monitor biological attributes of the user while the hearing devices are worn by the user. For example, a behind-the-ear (“BTE”) component of a hearing device may include a skin temperature sensor configured to monitor skin temperature of the user while the BTE component is worn behind the user's ear. However, skin temperature may significantly be affected by external influencing factors such as environmental conditions including ambient temperature, humidity, air velocity, etc. In view of this, a skin temperature sensor alone is inadequate for measuring a core body temperature of a user and often results in underestimated core body temperatures. Accordingly, there remains room to improve the manner in which hearing devices may be used to measure a core body temperature of a user.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary core body temperature processing system that may be implemented according to principles described herein.

FIG. 2 illustrates an exemplary implementation of the core body temperature processing system of FIG. 1 according to principles described herein.

FIG. 3 illustrates an exemplary flow diagram that may be implemented according to principles described herein.

FIGS. 4-5 illustrate exemplary configurations of hearing devices that may be implemented according to principles described herein.

FIGS. 6-7 illustrate exemplary configurations of hollow core optical waveguides that may be included in hearing devices according to principles described herein.

FIG. 8 illustrates an exemplary method according to principles described herein.

FIG. 9 illustrates an exemplary computing device according to principles described herein.

### DETAILED DESCRIPTION

Hearing devices, systems, and methods for measuring a core body temperature of a user are described herein. As will be described in more detail below, an exemplary hearing device may be configured to be inserted at least partially within an ear canal of a user of the hearing device and may comprise a first temperature sensor configured to detect ear canal temperature within the ear canal, a second temperature sensor configured to detect ambient temperature outside of the ear canal, and a processor. The processor may be configured to determine a work status of the user indicative of an activity level of the user and determine a core body temperature of the user based on the ear canal temperature detected by the first temperature sensor, the ambient temperature detected by the second temperature sensor, and the work status of the user.

By using hearing devices, systems and methods such as those described herein, it is possible to provide a non-invasive solution for an accurate determination of core body temperatures. For example, hearing devices such as those described herein may be configured with multiple temperature sensors that provide temperature readings that, in combination with other measured parameters (e.g., heart rate, work status, etc.), may be used to determine an accurate measure of a core body temperature of a user of the hearing devices. Moreover, hearing devices, systems, and methods such as those described herein may beneficially be configured to facilitate tracking and/or monitoring various conditions (e.g., fever, insomnia, fatigue, infection, etc.) that may be associated with a core body temperature of a user. In addition, hearing devices, systems and methods such as those described herein may provide one or more graphical user interface views that provide a user with information associated with physiological and/or pathological conditions associated with core body temperature. Other benefits of the systems and methods described herein will be made apparent herein.

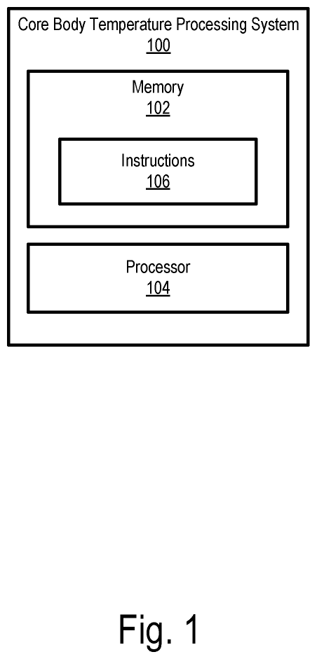

FIG. 1 illustrates an exemplary core body temperature processing system 100 (“system 100”) that may be implemented according to principles described herein. As shown, system 100 may include, without limitation, a memory 102 and a processor 104 selectively and communicatively coupled to one another. Memory 102 and processor 104 may each include or be implemented by hardware and/or software components (e.g., processors, memories, communication interfaces, instructions stored in memory for execution by the processors, etc.). In some examples, memory 102 and/or processor 104 may be implemented by any suitable computing device such as described herein. In other examples, memory 102 and/or processor 104 may be distributed between multiple devices and/or multiple locations as may serve a particular implementation. Illustrative implementations of system 100 are described herein.

Memory 102 may maintain (e.g., store) executable data used by processor 104 to perform any of the operations described herein. For example, memory 102 may store instructions 106 that may be executed by processor 104 to perform any of the operations described herein. Instructions 106 may be implemented by any suitable application, software, code, and/or other executable data instance.

Memory 102 may also maintain any data received, generated, managed, used, and/or transmitted by processor 104. Memory 102 may store any other suitable data as may serve a particular implementation. For example, memory 102 may store hearing loss profile data, user preference data, setting data, ambient temperature data, ear canal temperature data, heart rate data, work status information, machine learning data, graphical user interface content, and/or any other suitable data.

Processor 104 may be configured to perform (e.g., execute instructions 106 stored in memory 102 to perform) various processing operations associated with measuring a core body temperature of a user. For example, processor 104 may perform one or more operations described herein to determine a core body temperature of a user based on ear canal temperature detected by a first temperature sensor, ambient temperature detected by a second temperature sensor, and a work status of the user. These and other operations that may be performed by processor 104 are described herein.

As used herein, a “hearing device” may be implemented by any device or combination of devices configured to provide or enhance hearing to a user. For example, a hearing device may be implemented by a hearing aid configured to amplify audio content to a recipient, a sound processor included in a cochlear implant system configured to apply electrical stimulation representative of audio content to a recipient, a sound processor included in a stimulation system configured to apply electrical and acoustic stimulation to a recipient, or any other suitable hearing prosthesis. In some examples, a hearing device may be implemented by BTE housing configured to be worn behind an ear of a user. In some examples, a hearing device may be implemented by an in-the-ear (“ITE”) component configured to at least partially be inserted within an ear canal of a user. In some examples, a hearing device may include a combination of an ITE component, a BTE housing, and/or any other suitable component.

In certain examples, hearing devices such as those described herein may be implemented as part of a binaural hearing system. Such a binaural hearing system may include a first hearing device associated with a first ear of a user and a second hearing device associated with a second ear of a user. In such examples, the hearing devices may each be implemented by any type of hearing device configured to provide or enhance hearing to a user of a binaural hearing system. In some examples, the hearing devices in a binaural system may be of the same type. For example, the hearing devices may each be hearing aid devices. In certain alternative examples, the hearing devices may be of a different type. For example, a first hearing device may be a hearing aid and a second hearing device may be a sound processor included in a cochlear implant system.

In some examples, a hearing device may additionally or alternatively be implemented by one or more earbuds, one or more headphones, one or more hearables (e.g., smart headphones), and/or any other suitable device that may be used to facilitate a user perceiving sound. In such examples, the user may correspond to either a hearing impaired user or a non-hearing impaired user.

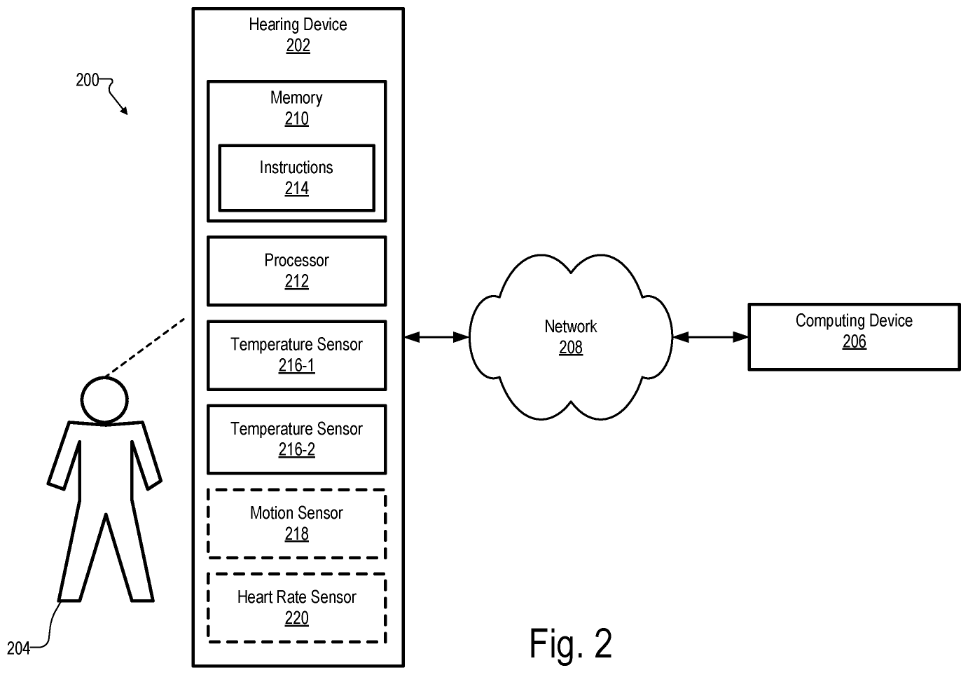

System 100 may be implemented in any suitable manner. For example, system 100 may be implemented by a hearing device and/or a computing device that is communicatively coupled in any suitable manner to the hearing device. To illustrate an example, FIG. 2 shows an exemplary implementation 200 in which system 100 may be provided in certain implementations. As shown in FIG. 2, implementation 200 includes a hearing device 202 that is associated with a user 204 and that is communicatively coupled to a computing device 206 by way of a network 208. User 204 may correspond to any individual that is a user of a hearing device such as described herein.

Hearing device 202 may correspond to any suitable type of hearing device such as described herein. Hearing device 202 may include, without limitation, a memory 210 and a processor 212 selectively and communicatively coupled to one another. Hearing device 202 may also include a housing configured to be inserted at least partially within an ear canal of a user of the hearing device. Such a housing may be configured in any suitable manner. For example, in certain implementations, the housing may form a portion of an ITE component that is at least partially inserted within an ear canal of a user. In certain examples, the housing may be custom formed for a particular user. Such customized housings may provide a more stable core body temperature measurement compared to standard housings under different environmental conditions. This may be achieved by customized housings creating a tight seal of the ear canal resulting in an increased thermal coupling to a wall of the ear canal and a decreased coupling to the environment. This in turn may mitigate the influence of environmental factors such as ambient temperature, humidity, solar irradiance, and/or air velocity in temperature readings from the ear canal. In addition, customized housings may minimize the effect that physical activity may have by reducing the amount of air exchange between the ear canal and the surrounding environment. In certain alternative examples, the housing may correspond to a standard housing that is configured to universally fit multiple different users. Exemplary housings are described herein.

Memory 210 and processor 212 may each include or be implemented by hardware and/or software components (e.g., processors, memories, communication interfaces, instructions stored in memory for execution by the processors, etc.). In some examples, memory 210 and processor 212 may be housed within or form part of a BTE housing. In some examples, memory 210 and processor 212 may be located separately from a BTE housing (e.g., in an ITE component). In some alternative examples, memory 210 and processor 212 may be distributed between multiple devices (e.g., multiple hearing devices in a binaural hearing system) and/or multiple locations as may serve a particular implementation.

Memory 210 may maintain (e.g., store) executable data used by processor 212 to perform any of the operations associated with hearing device 202. For example, memory 210 may store instructions 214 that may be executed by processor 212 to perform any of the operations associated with hearing device 202 assisting a user in hearing. Instructions 214 may be implemented by any suitable application, software, code, and/or other executable data instance.

Memory 210 may also maintain any data received, generated, managed, used, and/or transmitted by processor 212. For example, memory 210 may maintain any suitable data associated with a hearing loss profile of a user, ambient temperature data, ear canal temperature data, health profile information, etc. Memory 210 may maintain additional or alternative data in other implementations.

Processor 212 is configured to perform any suitable processing operation that may be associated with hearing device 202. For example, when hearing device 202 is implemented by a hearing aid device, such processing operations may include monitoring ambient sound and/or representing sound to user 204 via an in-ear receiver. Processor 212 may be implemented by any suitable combination of hardware and software.

As shown in FIG. 2, hearing device 202 further includes temperature sensors 216 (e.g., temperature sensors 216-1 and 216-2). Temperature sensor 216-1 may be configured to detect ear canal temperature within an ear canal of user 204. Temperature sensor 216-1 may be provided in a housing of hearing device 202 that is configured to be inserted at least partially in the ear canal of user 204. Temperature sensor 216-1 may be configured to detect the ear canal temperature at skin of the ear canal (e.g., at the ear canal wall). Temperature sensor 216-1 may also be configured to detect the ear canal temperature at a location within the ear canal remote from the skin. Assuming thermal equilibrium within the ear canal, the temperature measured at the skin may substantially correspond to the temperature measured remote from the skin. The ear canal temperature may thus also be denoted as a skin temperature within the ear canal. Ear canal temperature data indicative of the ear canal temperature provided by temperature sensor 216-1 may be accessed by processor 104, 212. Temperature sensor 216-1 may include any suitable type of temperature sensor as may serve a particular implementation. For example, temperature sensor 216-1 may include a flow temperature sensor, an infrared (“IR”) thermometer, and/or a contact temperature sensor. Temperature sensor 216-2 may be configured to detect ambient temperature outside of an ear canal of a user. Temperature sensor 216-2 may be positioned in any suitable manner with respect to hearing device 202 to detect ambient temperature. For example, temperature sensor 216-2 may be provided on a BTE component. Alternatively, temperature sensor 216-2 may be provided on a faceplate of an ITE component. Ambient temperature data indicative of the ambient temperature provided by temperature sensor 216-2 may be accessed by processor 104, 212. Temperature sensor 216-2 may include any suitable type of temperature sensor as may serve a particular implementation. Exemplary configurations of temperature sensors are described herein.

In certain examples, hearing device 202 may further include a motion sensor 218. Motion sensor 218 may be implemented by any suitable type of motion sensor as may serve a particular implementation. For example, motion sensor 218 may include an accelerometer and/or a gyroscope in certain examples. In some examples, motion sensor 218 may be implemented as an inertial measurement unit (IMU). Motion sensor 218 may be configured to detect motion data that may be used by system 100 to determine a work status of user 204. In certain examples, hearing device 202 may further include a heart rate sensor 220. Heart rate sensor 220 may be implemented by any suitable type of heart rate sensor as may serve a particular implementation. For example, heart rate sensor 220 may comprise a photoplethysmography (PPG) sensor and/or an electrocardiography (ECG) sensor in certain examples. Heart rate sensor 220 may be configured to detect heart rate data indicative of a heart rate of the user.

Motion sensor 218 and heart rate sensor 220 are shown in dashed lines in FIG. 2 because, in certain examples, a motion sensor and/or a heart rate sensor may be included in a device other than hearing device 202. For example, computing device 206 may correspond to a smartphone or a smartwatch with a motion sensor and/or a heart rate sensor. In such examples, hearing device 202 may access or otherwise obtain motion data from the smartphone or smartwatch to facilitate measuring the core body temperature of user 204. In some examples, the heart rate may be determined based on the ear canal temperature, the ambient temperature, and data including the motion data, e.g., the work status determined therefrom. In those examples, heart rate sensor 220 may not be required. In some examples, the heart rate may be determined based on the ear canal temperature, the ambient temperature, and data including the motion data and the heart rate data.

Computing device 206 may include or be implemented by any suitable hardware and/or software components (e.g., processors, memories, communication interfaces, instructions stored in memory for execution by the processors, etc.) and may include any combination of computing devices as may serve a particular implementation. In certain examples, computing device 206 may correspond to a smart charging device for hearing device 202, a laptop computer, a desktop computer, a tablet computer, and/or any other suitable computing device that may be configured to facilitate measuring a core body temperature of a user. In such examples, computing device 206 may be configured to perform any suitable operations such as those described herein to process temperature information, biological information, work status information, etc. to facilitate measuring a core body temperature of a user.

Network 208 may include, but is not limited to, one or more wireless networks (Wi-Fi networks), wireless communication networks, mobile telephone networks (e.g., cellular telephone networks), mobile phone data networks, broadband networks, narrowband networks, the Internet, local area networks, wide area networks, and any other networks capable of carrying data and/or communications signals between hearing device 202 and computing device 206. In certain examples, network 208 may be implemented by a Bluetooth protocol (e.g., Bluetooth Classic, Bluetooth Low Energy (“LE”), etc.) and/or any other suitable communication protocol to facilitate communications between hearing device 202 and computing device 206. Communications between hearing device 202, computing device 206, and any other device/system may be transported using any one of the above-listed networks, or any combination or sub-combination of the above-listed networks.

System 100 may be implemented by computing device 206 or hearing device 202. Alternatively, system 100 may be distributed across computing device 206 and hearing device 202, or distributed across computing device 206, hearing device 202, and/or any other suitable computing system/device.

Core body temperature is an important indicator of various medical conditions that a user of a hearing device may experience. For example, the core body temperature of a user may be indicative of fever, insomnia, infection, metabolic functionality, and/or depression. Typically, body temperature sensors implemented in wearable devices are configured to detect ear canal temperature. Even though ear canal temperature may provide relevant information for estimating a core body temperature, the use of single ear canal temperature measurements alone is insufficient and often results in underestimated or overestimated core body temperatures. This is because ear canal temperature may be significantly affected by external influencing factors such as environmental conditions including ambient temperature, humidity, and/or air velocity. Other techniques for measuring temperature (e.g., rectal, gastrointestinal, etc.) may provide relatively more accurate core body temperature measurements than ear canal temperature measurements but are invasive and inappropriate for wearable devices outside of a laboratory environment.

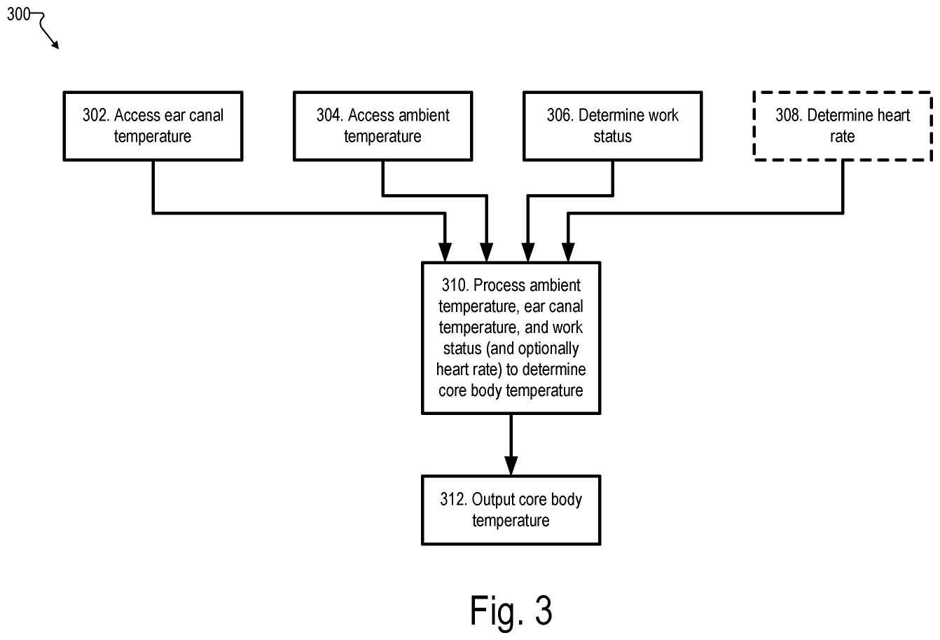

Hearing devices such as those described herein possess technical advantages for temperature monitoring due to the shared vasculature between the ear canal and the hypothalamus (the body's temperature control center) originating from the internal carotid artery. To that end, system 100 (e.g., processor 104, processor 212, etc.) may be configured to leverage information obtained by way of hearing devices such as those described herein to facilitate measuring the core body temperature of a user. This may be accomplished in any suitable manner. To illustrate, FIG. 3 shows a flow diagram 300 with various operations that may be performed by system 100 in measuring a core body temperature of a user. As shown in FIG. 3, at operation 302, system 100 may access ear canal temperature of a user. This may be accomplished in any suitable manner. For example, system 100 may obtain ear canal temperature data from temperature sensor 216-1.

At operation 304, system 100 may access ambient temperature in an environment of a user. This may be accomplished in any suitable manner. For example, system 100 may obtain ambient temperature data from temperature sensor 216-2.

At operation 306, system 100 may determine a work status of a user that is indicative of an activity level of the user. In certain examples, the work status may indicate whether the user is in a resting state or a physically active state. System 100 may determine the work status in any suitable manner. For example, system 100 may access information detected by a motion sensor (e.g., motion sensor 218) to determine the work status of the user. To illustrate an example, information received from a motion sensor may indicate that user 204 is currently walking based on a pattern of accelerations detected by the motion sensor. Accordingly, system 100 may determine that user 204 is at a relatively high level of activity. Alternatively, information received from the motion sensor may indicate that user 204 has not moved for a predefined amount of time. Accordingly, system 100 may determine that user 204 is at a relatively low level of activity. In some instances, the activity level and/or work status may be determined from information provided by a physiological sensor, e.g., in place of the motion data provided by motion sensor 218 or in addition to the motion data. For example, in cases where there is a high effort but a low motion signal, e.g., at a particular region of the user's body such as at the head level (e.g., during stationary cycling, high resistance training, leg work, isometric workouts, etc.) physiological sensor data may be gathered to estimate the activity level and/or work status, e.g., additionally or alternatively to the motion data. In some instances, the physiological sensor data may comprise heart rate data. For instance, the heart rate data may be provided by heart rate sensor 220. To illustrate, the heart rate may be evaluated relative to a heart rate threshold, and when the heart rate exceeds the threshold, the work status may be set to a predetermined value. The heart rate threshold may be set and/or predetermined by taking into account information about the user, e.g., the user's age. The heart rate threshold may also be set and/or predetermined by taking into account averaged heart rates of the user and/or other persons.

In certain examples, as illustrated in FIG. 3 as an operation 308 shown in dashed lines, system 100 may further determine a heart rate of the user to facilitate determining a core body temperature of a user. In such examples, system 100 may obtain the heart rate in any suitable manner. For example, in certain implementations, hearing device 202 may further include a heart rate sensor that may be configured to measure the heart rate of user 204 while hearing device 202 is worn by user 204. Alternatively, system 100 may obtain the heart rate from a device that is communicatively coupled to hearing device 202. For example, a smartwatch communicatively coupled to hearing device 202 may be configured to measure the heart rate of user 204 at any suitable time and provide such information to hearing device 202 to facilitate measuring the core body temperature of user 204.

At operation 310, system 100 may process the ambient temperature, the ear canal temperature, and the work status (and optionally the heart rate) to determine the core body temperature of the user. System 100 may be configured to perform operation 310 in any suitable manner. For example, in certain implementations, system 100 may be configured to use a model, such as a linear regression model or a nonlinear regression model, to estimate the core body temperature of the user. With such a linear regression model, a combination of non-invasive parameters associated with core body temperature may be used by system 100 to improve determining the core body temperature according to the following formula.

T_{c}=(a×HR)^{A}+(b×Tₑ)^{B}+(c×Tₐ)^{c}+(d×W)^{D}+e

Where “a,” “b,” “c,” “d,” and “e” are unknown coefficients and “A”, “B”, “C”, “D” are unknown exponents that may be determined empirically based on intended conditions and target age groups. In particular, “A”, “B”, “C”, “D” may be selected to be any value larger than zero, e.g., equal to one. “Tₑ” (>36.5° C.) is the insulated ear canal temperature. “Tₐ” is the ambient temperature (affecting the ear canal temperature and may therefore also be denoted as “skin microclimate temperature”) that may be measured by, for example, temperature sensor 216-2. “W” is the work status of the user (e.g., at rest=0; during exercise=1). The work status may be determined based on accelerometer data (e.g., captured by motion sensor 218) indicating whether the user is at rest or performing physical activity. The linear regression model may use the following ranges for HR, Tₑ, Tₐ; HR: 40-180 bpm; Tₑ: from 35-45° C.; and Tₐ: −10 to 45° C. An example range of the unknown coefficients may be as follows.
a(HR): 0.001 to 0.05
b(Tₑ): 0.02 to 0.8
c(Tₐ): −0.12 to 0.12
d(Work): −0.5 to 0.5
e: 10 to 40

At operation 312, system 100 may output the core body temperature. This may be accomplished in any suitable manner. For example, in certain implementations, system 100 may provide an audio notification to user 204 by way of hearing device 202 that informs user 204 of the core body temperature. Additionally or alternatively, system 100 may be configured to output the core body temperature to an external device for presentation to the user. For example, computing device 206 may correspond to a smart phone communicatively coupled to hearing device 202 by way of network 208. In such an example, system 100 may direct the smart phone to display a graphical user interface with information indicative of the core body temperature of the user.

In certain examples, system 100 may leverage an additional hearing device to facilitate determining a core body temperature of a user. In such examples, a first hearing device may be provided at a first ear of the user and a second hearing device may be provided at a second ear of the user. In such examples, both the first hearing device and the second hearing device may be configured in a manner similar to hearing device 202. As such, the first hearing device may be configured to determine a core body temperature value for the user and the second hearing device may be configured to determine an additional core body temperature value for the user. In such examples, system 100 may be configured to determine a core body temperature of the user based on the core body temperature value and the additional core body temperature value. This may be accomplished in any suitable manner. For example, system 100 may empirically and mathematically combine the core body temperature value and the additional core body temperature value into one output value to determine the core body temperature of the user. In certain examples, this may include system 100 determining an average core body temperature based on the core body temperature value and the additional core body temperature value.

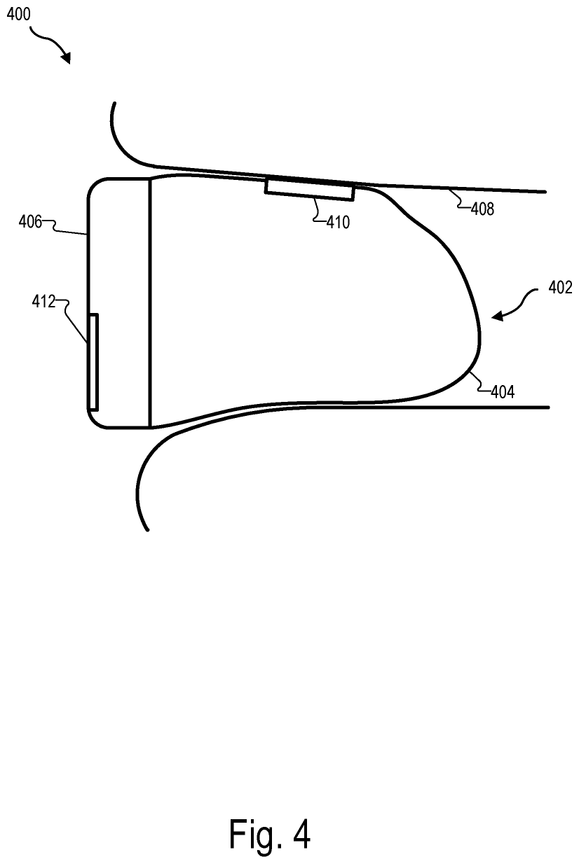

FIG. 4 shows an exemplary configuration 400 that a hearing device may have in certain examples. As shown in FIG. 4, a hearing device 402 includes a housing 404 and a faceplate 406. Housing 404 may be customized to fit within an ear canal 408 of a user. For example, housing 404 may be provided as a shell customized to an individual ear canal shape of the user. A first temperature sensor 410 is provided on a surface of housing 404 and is configured to detect ear canal temperature within ear canal 408. A second temperature sensor 412 is provided on an externally facing surface of faceplate 406 and is configured to detect ambient temperature outside of ear canal 408. In other examples, the hearing device may include a housing which is adapted for insertion in an ear canal of an average size and/or shape (e.g., a housing that is not customized to an individual ear canal). In such examples, the housing may be provided with a flexible sealing member, e.g., a dome, to provide for a seat of the hearing device inside the ear canal at the ear canal wall. The housing may further comprise an enclosure for accommodating hearing device components, e.g., a receiver, to which the sealing member is attached. The first temperature sensor 410 may be provided, for instance, at or in the enclosure for the hearing device components and/or at the sealing member.

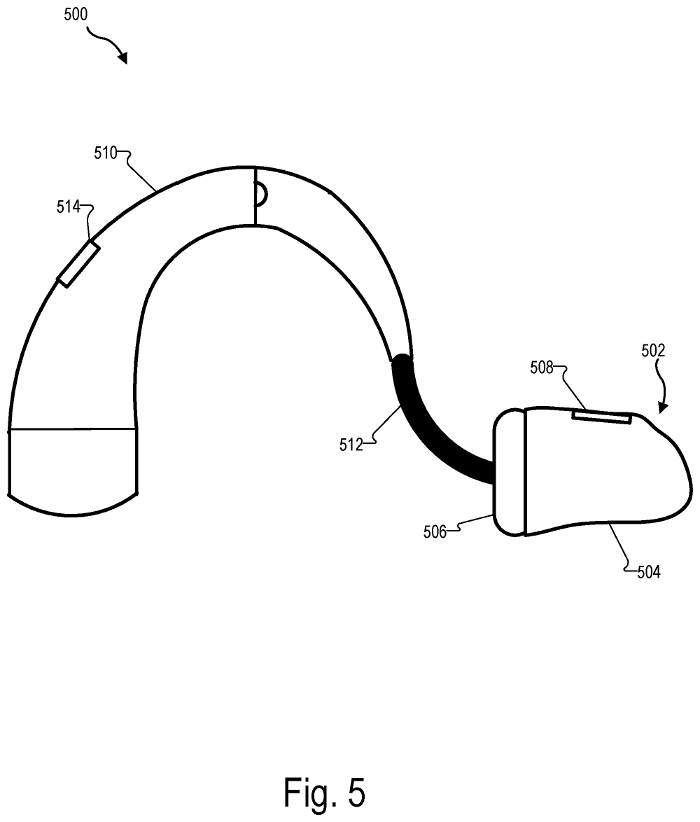

In certain examples, a temperature sensor that is configured to detect ambient temperature outside of an ear canal may be provided on a BTE component instead of a faceplate. To illustrate, FIG. 5 shows another exemplary configuration 500 that a hearing device may have in alternative examples. As shown in FIG. 5, configuration 500 includes an ITE component 502 including a housing 504, a faceplate 506, and a first temperature sensor 508 that may be configured to detect ear canal temperature within an ear canal of a user. As shown in FIG. 5, ITE component 502 is communicatively coupled to a BTE component 510 by way of a cable 512. BTE component 510 includes a second temperature sensor 514 that is configured to detect ambient temperature outside of the ear canal.

The position of second temperature sensor 514 on BTE component 510 is provided for illustrative purposes only. It is understood that second temperature sensor 514 may be provided at any suitable other position on BTE component 510 as may serve a particular implementation. In addition, in certain examples, BTE component 510 may include a plurality of temperature sensors configured to detect ambient temperature outside of the ear canal.

In certain examples, a housing of a hearing device may include a hollow core optical waveguide for a temperature sensor that is configured to detect ear canal temperature within an ear canal of a user. In such examples, the temperature sensor may correspond to an IR thermometer. The hollow core optical waveguide may be configured to enhance coupling efficiency of IR radiation from tissue in the ear canal to the IR thermometer while minimizing optical loss. Hollow core optical waveguides facilitate reliable and repeatable readings and detection of small variations in body temperature. In addition, hollow core optical waveguides may facilitate mitigating the influence of environmental factors such as ambient temperature, humidity, wind speed, etc.

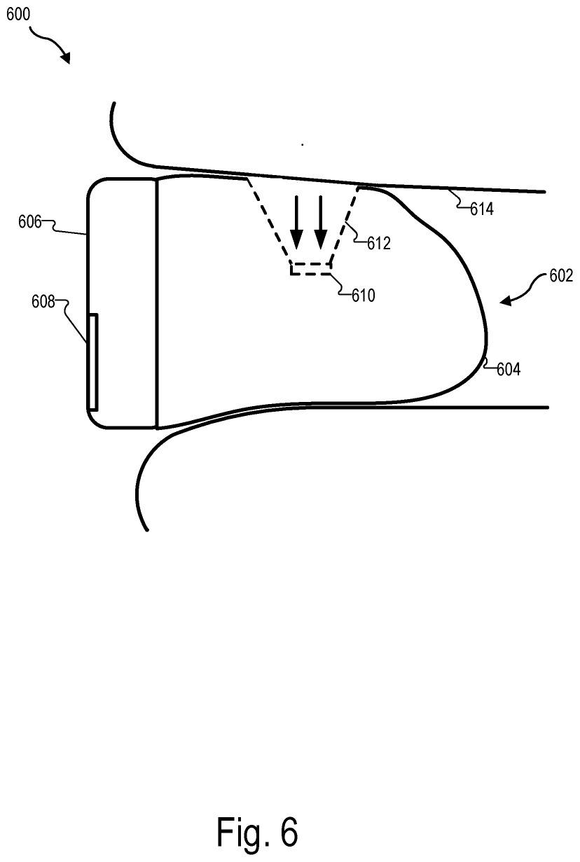

Hollow core optical waveguides such as those described herein may be configured in any suitable manner. In certain implementations, a hollow core optical waveguide may have a funnel shape. To illustrate, FIG. 6 shows an exemplary configuration 600 of an ITE component 602 that includes a housing 604 and a faceplate 606 that includes an ambient temperature sensor 608 provided on an externally facing surface thereof. An IR thermometer 610 is provided internally within housing 604 with a funnel shaped hollow core optical waveguide 612 configured to enhance coupling efficiency of IR radiation from tissue in ear canal 614 to IR thermometer 610. Funnel shaped hollow core optical waveguide 612 may be configured in any suitable manner such that when IR radiation is coupled from a wide end of waveguide 612 and propagates to a fine end of waveguide 612, the IR radiation output at the fine end is condensed. Waveguide 612 may have any suitable thickness as may serve a particular implementation. For example, in certain implementations, waveguide 612 may have a thickness of 0.3-0.5 mm.

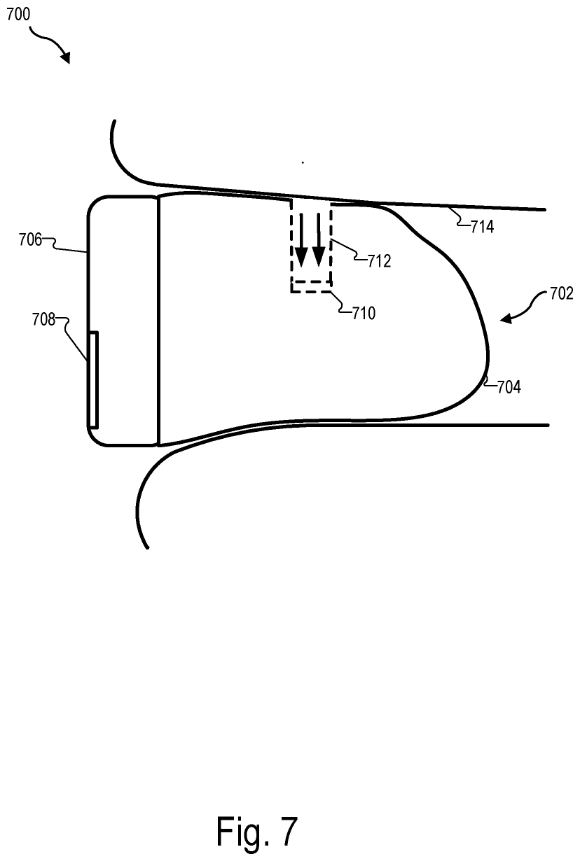

In certain alternative implementations, a hollow core optical waveguide may have a tube shape. To illustrate, FIG. 7 shows an exemplary configuration 700 of an ITE component 702 that includes a housing 704 and a faceplate 706 that includes an ambient temperature sensor 708 provided on an externally facing surface thereof. An IR thermometer 710 is provided internally within housing 704 with a tube shaped hollow core optical waveguide 712 configured to facilitate IR radiation propagating from a distal end of tube shaped hollow core optical waveguide 712 near skin of ear canal 714 to a proximal end of tube shaped hollow core optical waveguide 712 where IR thermometer 710 is located. Tube shaped hollow core optical waveguide 712 may have any suitable dimensions as may serve a particular implementation. For example, in certain implementations, tube shaped hollow core optical waveguide 712 may have a core inner diameter of 3 to 4 mm and a thickness of tube shaped hollow core optical waveguide 712 may be 0.3-0.5 mm.

In certain examples, a hollow core optical wave guide may be covered with a covering to provide ingress protection against earwax. For example, the wide end of a funnel shaped hollow core optical waveguide may be covered with such a covering. The covering may be formed of any suitable material as may serve a particular implementation. For example, the covering may be formed with a thin lens, glue, lacquer, or any other suitable substance to provide ingress protection against earwax.

In certain alternative implementations, system 100 may use a hybrid method to facilitate measuring a core body temperature of a user. Such a hybrid method makes use of the fact that the hypothalamus is the body's temperature control center, which regulates temperature by maintaining a fine balance between actual metabolic rate (heat production) and corresponding heat loss (e.g., through radiation, evaporation, convection/conduction). ITE devices/hearables possess technical advantages for temperature monitoring because the shared vasculature between the ear canal and the hypothalamus originating from the carotid artery may be used to accurately estimate a core body temperature of a user. In view of this, the hybrid method may estimate the core body temperature based on the arterial heat balance (ear canal shared vasculature with hypothalamus). A heat balance equation that may be used by system 100 to calculate absolute/relative core body temperature expresses the balance between the metabolic heat from the blood supply and the heat dissipated into the atmosphere to obtain thermal homeostasis.

The purpose of the thermoregulation system of the human body is to keep its constant core internal temperature, and for long exposures to a constant (moderate) thermal environment with a constant metabolic rate, the heat production is balanced with heat loss, while the heat storage within the human body is not significant. The produced heat is conducted by heat conduction through body tissues and bones and convection in blood vessels. Such heat flows through a series of thermal resistances from arteries as a proxy for core body temperature (the shared vasculature of the ear canal with the hypothalamus as body temperature control center) to the ear skin and from the ear skin to the surrounding environment.

Heat flow from the core arterial source to the surface of ear canal's skin can be described with the following heat transfer equation (based on electrical analog of heat flow and temperature principle):

q₁=1/R₁×(T_{c}−Tₑ)
where “q₁” is heat flow, “Tₑ” and “T_{c}” are the ear canal and core temperatures, respectively, and “R1” is thermal resistance. Heat loss of the ear canal to the environment (e.g., as a result of temperature gradient from the ear canal and ambient temperature) may be calculated with the following equation (based on electrical analog of heat flow and temperature principle):

q₂=1/R₂×(Tₑ−Tₐ)
where “q2” is heat flow, “Tₑ” and “Tₐ” are the ear canal and ambient temperatures, respectively, and “R₂” is thermal resistance. At equilibrium the heat flux from the blood supply (q1) and the heat dissipated into the atmosphere (q2) are equal to obtain thermal homeostasis:

q₁=q₂

The absolute core temperature may be determined as:

T_{c}=k×(Tₑ−Tₐ)+Tₐ

In the above expression, “k”, with (R₁+R₂)/R₁, is an unknown coefficient which may be defined empirically over a range of patients, situations, and also depending on the material property of the earpiece. “Tₐ” is the ambient temperature (“skin microclimate temperature”). After sensing ear canal temperature and ambient temperature, the absolute core temperature may be calculated. In certain examples, “k” may be written as (1+h/pc), where “h” is the heat transfer coefficient, “p” the blood perfusion rate per unit area at the skin and “c” is the specific heat of blood.

The hybrid method described above may have the following edge cases:

If h=0, which means the heat loss is zero, then T_{c}=Tₑ. That is, ear canal temperature is the same as core body temperature.

If p=∞, which means the perfusion is infinite, then T_{c}=Tₑ.

If Tₐ=T_{c}, which means that ambient temperature is near core body temperature, then T_{c}=Tₑ.

The parameter “k” may be estimated to be between −10 and 10. The parameter “k” may be estimated in any suitable manner. For example, the estimation of “k” may be based on a rational selection of possible temperatures of the body, the ambient environment, and/or the ear canal, e.g., skin, of the user.

In certain examples, system 100 may calibrate either one or both of the temperature sensors (e.g., temperature sensors 216-1 and 216-2) to facilitate measuring a core body temperature of a user. The temperature sensors may be calibrated in any suitable manner. For example, in certain implementations, both temperature sensors may be calibrated such that no further calibration is necessary. In certain alternative implementations, one of the temperature sensors may be calibrated. The other temperature sensor may then be calibrated during a situation where both of the temperature sensors are away from the user (e.g., while charging in a charging box) and experiencing the same temperature. In certain alternative examples, none of the temperature sensors of the hearing device may be calibrated initially but a calibrated temperature sensor may be provided in an additional device, e.g., an auxiliary device. For example, a charger base may include a calibrated temperature sensor. In such an example, the temperature sensor of the hearing device may become calibrated when they are associated with the additional device (e.g., during charging). In certain implementations, such an additional device may include a UVC light for disinfection purposes, a dry cleaning capability, a pressure sensor to facilitate estimating the temperature, a humidity sensor, a setup for acoustical self-tests of the hearing device, a GPS sensor that may be used to track a location of the additional device if lost, and/or any other suitable component. In some instances, processor 104, 212 can be configured to determine a situation in which first temperature sensor 216-1 and second temperature sensor 216-2 are exposed to an equal temperature, e.g., ambient temperature Tₐ, and to calibrate first temperature sensor 216-1 and second temperature sensor 216-2, e.g., the ear canal temperature data provided by temperature sensor 216-1 and the ambient temperature data provided by temperature sensor 216-2, relative to the equal temperature. The situation in which first temperature sensor 216-1 and second temperature sensor 216-2 are exposed to the equal temperature may comprise a situation in which the hearing device is removed from the ear of the user. The situation in which first temperature sensor 216-1 and second temperature sensor 216-2 are exposed to the equal temperature may also comprise a situation in which the hearing device is inserted and/or positioned and/or attached and/or coupled to the additional device. In some instances, the additional device may be a charger and processor 104, 212 may be configured to determine the situation in which first temperature sensor 216-1 and second temperature sensor 216-2 are exposed to an equal temperature based in whether the hearing device is inserted and/or connected to the charger and/or being charged. In some instances, the additional device may be a disinfection device and processor 104, 212 may be configured to determine the situation in which first temperature sensor 216-1 and second temperature sensor 216-2 are exposed to an equal temperature based in whether the hearing device is exposed to radiation for the disinfection, such as UVC light, and/or the hearing device is inserted and/or connected to the disinfection station.

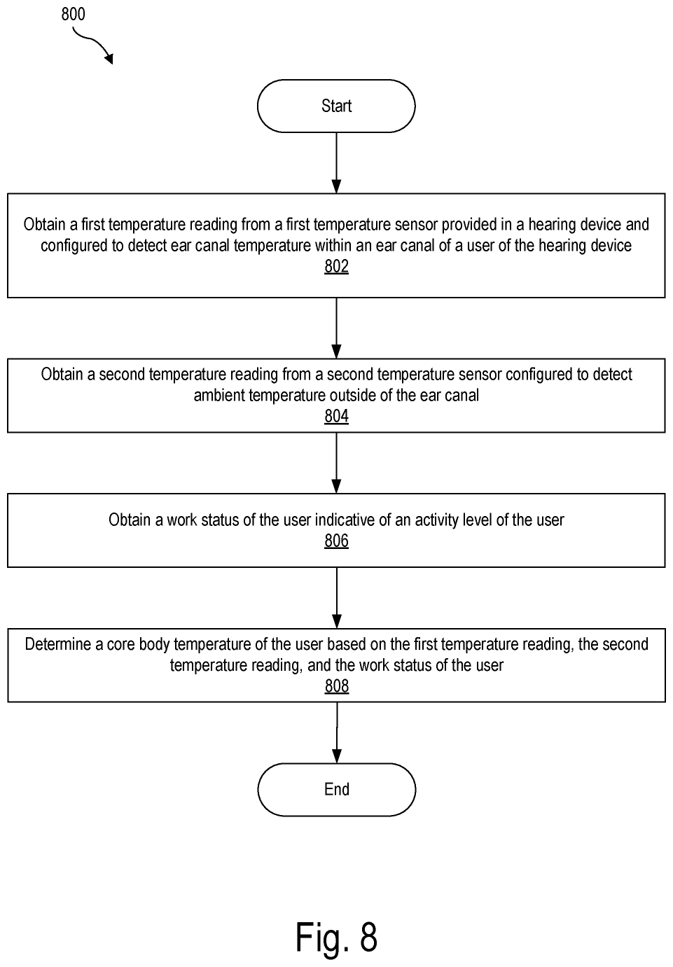

FIG. 8 illustrates an exemplary method 800 for measuring a core body temperature of a user according to principles described herein. While FIG. 8 illustrates exemplary operations according to one embodiment, other embodiments may omit, add to, reorder, and/or modify any of the operations shown in FIG. 8. One or more of the operations shown in FIG. 8 may be performed by core body temperature processing system 100, a hearing device such as hearing device 202, a computing device such as computing device 206, an additional computing device communicatively coupled to computing device 206 and/or hearing device 202, any components included therein, and/or any combination or implementation thereof.

At operation 802, a core body temperature processing system such as core body temperature processing system 100 may obtain a first temperature reading from a first temperature sensor provided in a hearing device and configured to detect ear canal temperature within an ear canal of a user of the hearing device. Operation 802 may be performed in any of the ways described herein.

At operation 804, the core body temperature processing system may obtain a second temperature reading from a second temperature sensor configured to detect ambient temperature outside of the ear canal. Operation 804 may be performed in any of the ways described herein.

At operation 806, the core body temperature processing system may obtain a work status of the user indicative of an activity level of the user. Operation 806 may be performed in any of the ways described herein.

At operation 808, the core body temperature processing system may determine a core body temperature of the user based on the first temperature reading, the second temperature reading, and the work status of the user. Operation 808 may be performed in any of the ways described herein.

In some examples, a computer program product embodied in a non-transitory computer-readable storage medium may be provided. In such examples, the non-transitory computer-readable storage medium may store computer-readable instructions in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g., a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

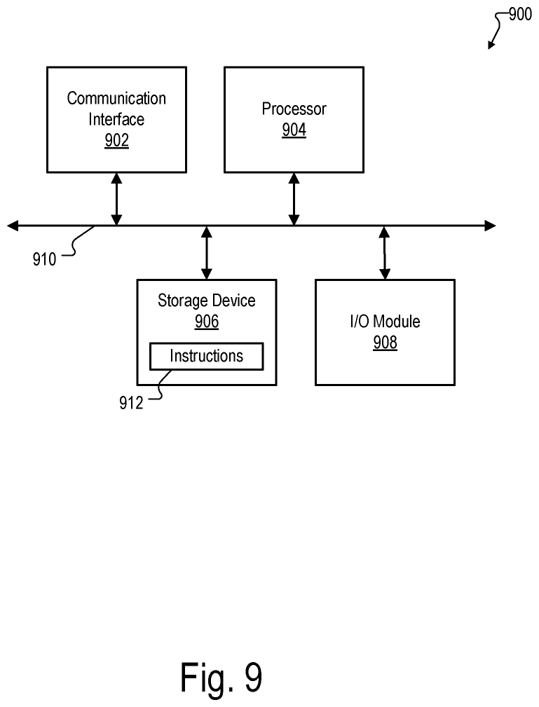

FIG. 9 illustrates an exemplary computing device 900 that may be specifically configured to perform one or more of the processes described herein. As shown in FIG. 9, computing device 900 may include a communication interface 902, a processor 904, a storage device 906, and an input/output (“I/O”) module 908 communicatively connected one to another via a communication infrastructure 910. While an exemplary computing device 900 is shown in FIG. 9, the components illustrated in FIG. 9 are not intended to be limiting. Additional or alternative components may be used in other embodiments. Components of computing device 900 shown in FIG. 9 will now be described in additional detail.

Communication interface 902 may be configured to communicate with one or more computing devices. Examples of communication interface 902 include, without limitation, a wired network interface (such as a network interface card), a wireless network interface (such as a wireless network interface card), a modem, an audio/video connection, and any other suitable interface.

Processor 904 generally represents any type or form of processing unit capable of processing data and/or interpreting, executing, and/or directing execution of one or more of the instructions, processes, and/or operations described herein. Processor 904 may perform operations by executing computer-executable instructions 912 (e.g., an application, software, code, and/or other executable data instance) stored in storage device 906.

Storage device 906 may include one or more data storage media, devices, or configurations and may employ any type, form, and combination of data storage media and/or device. For example, storage device 906 may include, but is not limited to, any combination of the non-volatile media and/or volatile media described herein. Electronic data, including data described herein, may be temporarily and/or permanently stored in storage device 906. For example, data representative of computer-executable instructions 912 configured to direct processor 904 to perform any of the operations described herein may be stored within storage device 906. In some examples, data may be arranged in one or more databases residing within storage device 906.

I/O module 908 may include one or more I/O modules configured to receive user input and provide user output. 1/O module 908 may include any hardware, firmware, software, or combination thereof supportive of input and output capabilities. For example, I/O module 908 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 908 is configured to provide graphical data to a display for presentation to a user. The graphical data may be representative of one or more graphical user interfaces and/or any other graphical content as may serve a particular implementation.

In some examples, any of the systems, hearing devices, computing devices, and/or other components described herein may be implemented by computing device 900. For example, memory 102 and/or memory 210 may be implemented by storage device 906, and processor 104 and/or processor 212 may be implemented by processor 904.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A hearing device configured to be at least partially inserted into an ear canal of a user, the hearing device comprising:
a first temperature sensor configured to detect an ear canal temperature within the ear canal;
a second temperature sensor configured to detect ambient temperature outside of the ear canal; and
a processor configured to:
determine a work status of the user indicative of an activity level of the user; and
determine a core body temperature of the user based on the ear canal temperature detected by the first temperature sensor, the ambient temperature detected by the second temperature sensor, and the work status of the user.

2. The hearing device of claim 1, wherein the processor is further configured to determine the core body temperature of the user based on a heart rate of the user.

3. The hearing device of claim 1, wherein the determining of the core body temperature includes using a linear regression model to estimate the core body temperature.

4. The hearing device of claim 1, wherein the first temperature sensor is a flow temperature sensor, an infrared (IR) thermometer, or a contact temperature sensor.

5. The hearing device of claim 1, wherein:
the first temperature sensor is an infrared (IR) thermometer; and
the hearing device further includes a housing a having a hollow core optical waveguide configured to enhance coupling efficiency of IR radiation from tissue in the ear canal to the IR thermometer.

6. The hearing device of claim 5, wherein the hollow core optical waveguide has a funnel shape.

7. The hearing device of claim 5, wherein the hollow core optical waveguide has a tube shape.

8. The hearing device of claim 1, wherein:
the hearing device further includes a housing having a faceplate that is configured to face outside of the ear canal when the hearing device is worn by the user; and
the second temperature sensor is positioned on the faceplate.

9. The hearing device of claim 1, further comprising a behind-the-ear (BTE) component,
wherein the second temperature sensor is positioned on the BTE component.

10. The hearing device of claim 1, wherein the hearing device is customized to fit at least partially within the ear canal of the user.

11. The hearing device of claim 1, wherein the processor is further configured to direct the hearing device to provide an audio notification that informs the user of the core body temperature.

12. The hearing device of claim 1, wherein the processor is further configured to:
determine a situation in which the first temperature sensor and the second temperature sensor are exposed to an equal temperature; and
calibrate the first temperature sensor and the second temperature sensor relative to the equal temperature.

13. The hearing device of claim 1, further comprising a motion sensor,
wherein the determining of the work status is based on information received from the motion sensor.

14. A system comprising:
a hearing device configured to be at least partially inserted into an ear canal of a user, the hearing device comprising:
a first temperature sensor configured to detect ear canal temperature within the ear canal; and
a second temperature sensor configured to detect ambient temperature outside of the ear canal; and
a processor configured to:
determine a work status of the user indicative of an activity level of the user; and
determine a core body temperature value of the user based on the ear canal temperature detected by the first temperature sensor, the ambient temperature detected by the second temperature sensor, and the work status of the user.

15. The system of claim 14, further comprising an external device that is communicatively coupled with the hearing device and is configured to present information associated with the core body temperature to the user.

16. The system of claim 15, wherein the processor is included in the external device that is communicatively coupled with the hearing device.

17. The system of claim 14, further comprising an additional hearing device configured to be at least partially inserted into an additional ear canal of a user, the additional hearing device comprising:
a third temperature sensor configured to detect ear canal temperature within the additional ear canal; and
a fourth temperature sensor configured to detect ambient temperature outside of the additional ear canal,
wherein the processor is further configured to determine an additional value of the work status of the user indicative of an activity level of the user, and to determine an additional core body temperature value of the user based on the ear canal temperature detected by the third temperature sensor, the ambient temperature detected by the fourth temperature sensor, and the additional value of the work status.

18. The system of claim 17, wherein the processor is further configured to determine a core body temperature of the user based on the core body temperature value and the additional core body temperature value.

19. A method comprising:
obtaining, by a core body temperature processing system, a first temperature reading from a first temperature sensor provided in a hearing device and configured to detect ear canal temperature within an ear canal of a user of the hearing device;
obtaining, by the core body temperature processing system, a second temperature reading from a second temperature sensor configured to detect ambient temperature outside of the ear canal;
obtaining, by the core body temperature processing system, a work status of the user indicative of an activity level of the user; and
determining, by the core body temperature processing system, a core body temperature of the user based on the first temperature reading, the second temperature reading, and the work status of the user.

20. The method of claim 19, further comprising obtaining, by the core body temperature processing system, a heart rate of the user,
wherein the core body temperature of the user is further determined based on the heart rate of the user.
